(19) United States
(12) Patent Application Publication
(10) Pub. No.: US 2006/0116695 A1
(43) Pub. Date: Jun. 1, 2006

(54) Implant anchor and methods of use

(76) Inventor: Andrew I. Poutiasrine, San Anselmo, CA (US)

Correspondence Address: DURECT CORPORATION, 10240 BUBB ROAD, CUPERTINO, CA, 95014, US

(21) Appl. No.: 10/537,442

(22) PCT Filed: Dec. 4, 2003
(86) PCT No.: PCT/US03/38549

§ 371 Date: Jun. 2, 2005

Related U.S. Application Data
(60) Provisional application No. 60/430,760, filed on Dec. 4, 2002.

(51) Int. Cl. A61F 2/02

(52) U.S. Cl. 606/151; 623/23.74

(57) ABSTRACT

An implant anchor that holds an implant in place by fibrotic encapsulation or inclusion of surrounding tissue into the anchor. The apparatus of the invention comprises an anchor configured to couple to an implant and which has at least one fibrous surface or portion capable of becoming fibrously encapsulated or included by surrounding tissue. The methods of the invention comprise providing an anchor having at least one porous surface or portion thereon, attaching the anchor to an implant, positioning the implant and attached anchor in a patient incision, and fibrotically encapsulating the anchor.

### FIELD OF THE INVENTION

This invention pertains generally to implantable drug delivery systems, and more particularly to a porous catheter anchor that provides fibrotic binding to hold a catheter in place.

### BACKGROUND OF THE INVENTION

Implantable drug delivery devices are increasingly used as therapeutic tools for treatment of a variety of conditions and diseases, especially where a prolonged period of therapy is required. Implantable drug delivery devices avoid patient inconvenience and discomfort associated with administration of multiple doses of an agent, and further provide for enhanced therapeutic benefits due to avoidance of bolus doses, improved patient compliance with dosage regimens, and providing generally a constant blood serum level of delivered drugs.

Various implantable drug delivery systems have been developed using different technologies to accomplish movement of drug (typically in a drug formulation) from within a reservoir in the device through an exit port or orifice to a treatment site in the subject. These delivery technologies have been based on, inter alia, diffusive, erodible, and convective mechanisms. Exemplary delivery systems employing convection include, but are not limited to, electromechanical pumps, osmotic pumps, electro-osmotic pumps, electrochemical pumps, hydrolytic systems, piezoelectric pumps, elastomeric pumps, vapor pressure pumps, and electrolytic pumps.

The implanted catheter must remain in place without longitudinal or transverse migration of the catheter following the implant procedure. The implanted catheter is particularly prone to movement in a longitudinal direction due to inadvertent pulling on the catheter during normal patient movement. Movement of the catheter can result in displacement of the catheter tip or disconnection of the catheter from the implanted pump such that drug delivery does not occur at the intended location, or reconfiguration or re-positioning of the catheter in a manner which otherwise interrupts or interferes with the intended drug delivery therapy for a patient.

One common technique for prevention of catheter movement is the use of catheter anchors made of a biocompatible polymer material such as silicone. The anchors are attached to the catheter and are sutured into place to hold the catheter in a desired location. The use of such anchors however is intrusive and requires additional surgical procedures, with relatively large incisions required to accommodate the anchor, and suturing of the anchors to hold them in place. Barbed structures have been utilized on catheters to avoid the need for additional suturing. The use of barbed catheters, however, complicates the initial positioning of the catheter during the implant procedure, as the barbs tend to catch on tissue and interfere with the movement of the catheter into its desired position.

There is accordingly a need for an anchor for catheters and other implanted devices which does not require additional incisions or suturing to hold the anchor in place, and which is quick and easy to position during implanting. The present invention satisfies these needs, as well as others, and generally overcomes the deficiencies found in the background art

### SUMMARY OF THE INVENTION

The invention provides an implant anchor that holds an implant such as a catheter in place by fibrotic encapsulation or inclusion of surrounding tissue into the anchor. The apparatus of the invention comprises, in general terms, an anchor configured to couple to an implant and which has a fibrous surface or portion capable of undergoing fibrous encapsulation or inclusion by tissue adjacent to, surrounding, or otherwise associated with the implant. In certain embodiments the anchor may be of tubular configuration, with an inner surface configured to fit over the outer wall of the implant, and with a fibrous outer surface configured to intermesh with surrounding tissue following implantation. The anchor may be used with a catheter and positioned adjacent to an end of the catheter or elsewhere thereon. The anchor may be capable of coupling to a catheter at more than one location such that a loop is formed in a catheter for additional anchoring capability. In other embodiments, the porous anchor may comprise two or more tubular sections joined together, with each tubular section configured to engage different portions of an implant.

The methods of the invention comprise providing an anchor having a porous surface thereon, attaching the anchor to an implant, and positioning the implant and attached anchor in a patient incision. The methods may additionally comprise fibrotically encapsulating the anchor after implantation to hold the implant in place. In certain embodiments the method of the invention may comprise coupling a first portion of a fibrous anchor to a first portion of an implant and coupling a second portion of a fibrous anchor to a second portion of an implant, positioning the implant and attached anchor in a patient incision, and fibrotically encapsulating the anchor in surrounding tissue. In still other embodiments, the method may comprise coupling a first fibrous anchor to a first portion of an implant, coupling a second fibrous anchor to a second portion of an implant, implanting the implant and attached fibrous anchors into tissue, and fibrotically encapsulating the anchors with surrounding tissue.

### BRIEF DESCRIPTIONS OF THE DRAWINGS

The present invention will be more filly understood by reference to the following drawings, which are for illustrative purposes only.

FIG. 1 is a perspective view of a porous catheter anchor in accordance with the invention.

FIG. 2 is a perspective view of the porous catheter anchor of FIG. 1 shown on a catheter.

FIG. 3 is a perspective view of an alternative embodiment porous catheter anchor in accordance with the invention.

FIG. 4 is a perspective view of the porous catheter anchor of FIG. 3 shown on a catheter.

### DETAILED DESCRIPTION OF THE INVENTION

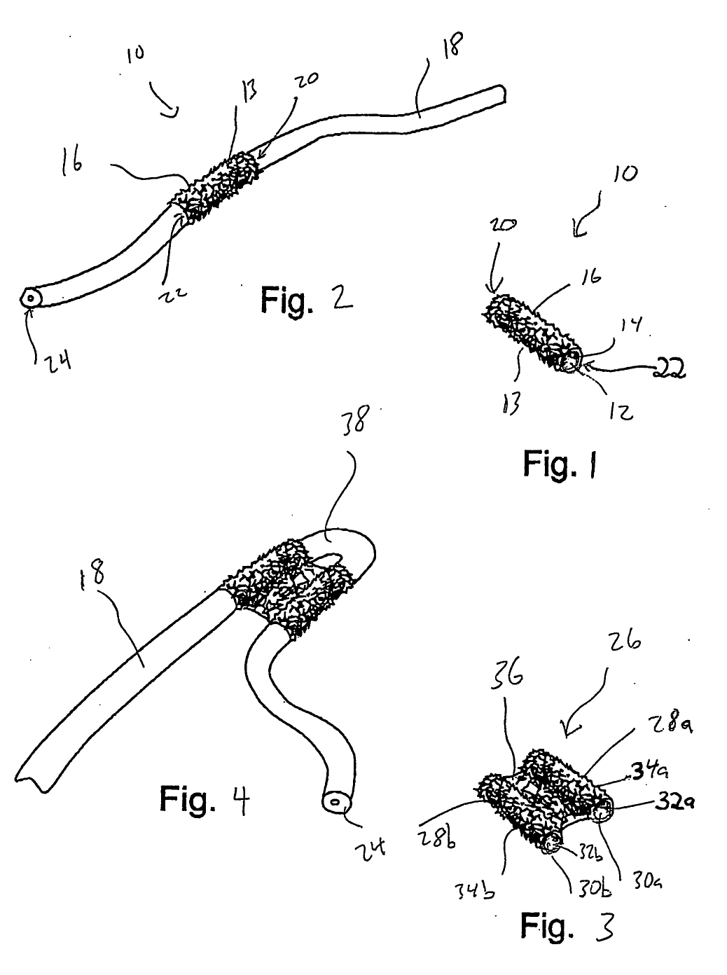

Referring more specifically to the drawings, for illustrative purposes the present invention is embodied in the apparatus and method shown generally in FIG. 1 through FIG. 4. It will be appreciated that the apparatus may vary as to configuration and as to details of the parts, and that the method may vary as to details and the order of the steps, without departing from the basic concepts as disclosed herein. The invention is disclosed primarily in terms of use with an implanted catheter. The invention may be used to anchor a variety of surgically implants or implanted devices, and use of the subject implant anchor apparatus and methods is intended to encompass such implants.

The definitions herein are provided for reason of clarity, and should not be considered as limiting. The technical and scientific terms used herein are intended to have the same meaning as commonly understood by one of ordinary skill in the art to which the invention pertains.

It should be noted that, as used in this specification and the appended claims, the singular forms “a,” “an” and “the” include plural referents unless the content clearly dictates otherwise. Thus, for example, reference to “a catheter” includes one or more such catheters, and “an anchor” includes one or more anchors, and the like.

As used herein, “patient”, individual”, “host” and “subject” and grammatical equivalents thereof means a member or members of any mammalian or non-mammalian species that may have a surgical implant or be in need of a surgical implant.

Referring now to FIG. 1 and FIG. 2, there is shown an implant anchor apparatus 10 in accordance with the invention. Anchor 10 is shown as having a tubular or cylindrical body 13 with a bore or channel 12 extending therethrough such that anchor 10 has an inner surface 14 and an outer surface 16. The dimensions and shape of bore 12 and inner surface 14 of anchor may be varied as required to engage or couple to a variety of surgical implants.

As shown in FIG. 1 and FIG. 2 and described further below, anchor 10 is configured for use with an implantable catheter 18. Catheter 18 may be a resilient or flexible elongated tube of the type commonly used for drug delivery to a selected site. The inner diameter of anchor 10 as defined by inner surface 14 is structured and configured to conform to the outer diameter of catheter 18 and to allow anchor 10 to be secured to catheter 18 when catheter 18 is engaged in bore 12. Anchor 10 may be coupled or attached to catheter 18 by use of a biocompatible adhesive material (not shown) that adheres the inner surface of 14 of anchor to catheter 18, by use of clips, clamps or fasteners (also not shown), by friction between the inner surface 14 of anchor 10 and catheter, or by other coupling means.

Anchor 10 comprises a porous or fibrous material that is capable of becoming fibrotically encapsulated by surrounding tissue after implantation. In other words, the material of anchor 10 or at least a portion of anchor 10 is suitably porous or fibrous such that a plurality of interstices (not shown) are defined, and human or other implant host tissue can penetrate into the interstices and enmesh with the material of the anchor 10 to hold the anchor 10 in place within the surrounding tissue. In the embodiment shown in FIG. 1 and FIG. 2, outer surface 16 of anchor 10 provides a fibrous or porous portion or section suitable for fibrotic encapsulation when anchor 10 is coupled to or otherwise associated with an implanted item such as catheter 18. Anchor 10 may be fabricated from a single integral piece of fibrous or porous material that is woven or bound together to define anchor 10. In other embodiments, anchor 10 may comprise a fibrous outer portion adjacent outer surface 16 that is adhered to a non-fibrous inner portion adjacent inner surface 14. Anchor 10 is shown as having fibrous material associated with the entire outer surface 16. Anchor 10 may, in some embodiments, include fibrous material on only a portion or portions of outer surface 16.

The fibrous material of anchor 10 may comprise any biologically compatible fibrous material that is capable of becoming fibrotically encapsulated in tissue. Various fibrous materials suitable for implantation are known and include, for example, polyester, polyamide, nylon, polyether, fluorocarbon, or other natural or man-made polymeric materials capable of forming fibers and fibrous networks. DACRON® polyester of Du Pont Inc. and KODEL® polyester of Kodak Inc. are, for example, two well known polyester fiber materials that are usable as medical implant materials. Various other fibrous materials suitable for use with the will suggest themselves to those skilled in the art.

In the methods of the invention, anchor 10 is suitably coupled to a catheter 18 or other implanted item, Anchor 10 includes first and second ends 20, 22, and is coupled to catheter 18 by inserting an end 24 of catheter 18 into the bore 12 of anchor 10 at end 20. The catheter end 24 is pushed or otherwise manipulated through bore 12 until catheter end 24 exits end 22 of anchor 10. Once thus inserted through bore 12, the outer surface of catheter 18 frictionally interacts with the inner surface 14 of anchor to couple or attach anchor 10 to catheter 18. In some embodiments of the invention, an adhesive may be used to adhere anchor 10 to catheter 18. The adhesive may be applied to the inner surface 14 of anchor prior to insertion of catheter 18 into bore 12, or the adhesive may be applied through the fibrous material of anchor 10 after anchor 10 has been suitably positioned on catheter 18. Various biologically compatible adhesives are commercially available and may be used to secure anchor 10 onto catheter 18 or other implantable device.

Once the anchor 10 has been suitably coupled to catheter 18, anchor and catheter 18 are implanted within an incision (not shown) in a patient or host, and the incision is sutured closed in a conventional manner. Following implantation, the tissue surrounding anchor 10 penetrates into the outer surface 16 of anchor 10 and into the interstices of the fibrous material of anchor such that the anchor becomes fibrotically encapsulated within the surround tissue to hold the anchor 10, and the attached catheter 18, in place within the incision. Fibrotic encapsulation of anchor 10 by surround tissue occurs relatively rapidly, with some fibrotic encapsulation occurring within a few hours, and complete fibrotic encapsulation of anchor 10 occurring within a few days.

Once fibrotic encapsulation in the above manner has occurred, the anchor 10 and catheter 18 are securely held at the desired location within the implant incision. Inadvertent or pressure or pulling on the catheter 18 thus will not result in undesirable repositioning catheter 18 or catheter end 24 after implantation. Anchor 10 and catheter 18 may be removed or detached from surrounding tissue following fibrotic encapsulation by creating a new incision in a conventional manner and removing the catheter 18 and attached anchor 10 from the incision.

Multiple anchors 10 may be coupled to multiple portions of a single catheter 18 to provide additional anchoring capability. Thus, a first anchor 10 may be coupled to catheter 18 near one end thereof to maintain the catheter end at a desired drug delivery site, while a second anchor 10 is coupled near the other end to prevent disconnection of the catheter from an implanted drug pump or reservoir (not shown). Anchor 10 may be elongated or truncated in shape to increase or reduce the portion of fibrous surface area available for fibrotic encapsulation by surrounding tissue.

Referring now to FIG. 3 and FIG. 4 there is shown an alternative embodiment 26 of an implant anchor in accordance with the invention. The anchor 26 comprises first and second tubular portions 28a, 28b each having a corresponding bore or channel 30a, 30b extending therethrough to define inner surfaces 32a, 32b. Tubular portions 28a, 28b respectively include fibrous or porous outer surfaces 34a, 34b capable of undergoing fibrotic encapsulation of surrounding tissue in the manner described above. Tubular portions 28a, 28b are joined together by a bridge or connecting region 36. The tubular portions 28a, 28b and connecting region 36 of anchor 26 may comprise integral portions of a single piece of fibrous material such as DACRON® polyester.

In using the anchor 26, catheter end 24 is inserted through both bores 30a, 30b of portions 28a, 28b such that catheter 18 is engaged in both bores 30a, 30b and a loop or fold 38 is defined in catheter 18 as shown in FIG. 4. Anchor 26 thus is coupled to catheter 18 at two points or locations to define loop 38. The anchor 26 and attached catheter 18 is implanted within an incision in the manner described above. The combined fibrous surface areas of the outer surfaces 34a, 34b of tubular portions 28a, 28b and connecting region 36 provide a relatively large area or region of fibrous material to provide a greater amount of fibrotic encapsulation by surround tissue, and thus greater anchoring effect, than is provided by the anchor 10 described above. The fold or loop 38 in catheter 18 can also act as an anchor within the surrounding tissue. The dual tubular portions 28a, 28b, in coupling to different portions of catheter 18, also provide stronger coupling between the anchor 26 and catheter 18.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An anchor apparatus comprising at least one porous section, said anchor apparatus configured to couple to an implant, said porous section capable of becoming fibrotically encapsulated in tissue.

2. The anchor apparatus of claim 1, further comprising a first porous section configured to couple to a first portion of an implant, and a second porous section configured to couple to a second portion of an implant.

3. The implant anchor apparatus of claim 1, further comprising a first tubular component, said porous section associated with an outer surface of said first tubular section, said tubular component including an inner surface configured to engage said implant.

4. The implant anchor apparatus of claim 3, further comprising a second tubular component joined to said first tubular component, said second tubular component including a porous section on an outer surface thereof and an inner surface configured to engage said implant, said porous section on said second tubular component capable of becoming fibrotically encapsulated in tissue.

5. An implant anchor apparatus comprising:
(a) a tubular section of fibrous material;
(b) said tubular section having an inner surface configured to fit adjacent implant; and
(c) said fibrous material capable of becoming fibrotically encapsulated in tissue.

6. The implant anchor apparatus of claim 5, wherein said fibrous material comprises a polyester.

7. The implant anchor apparatus of claim 5, wherein said fibrous material comprises a nylon.

8. The implant anchor apparatus of claim 5, wherein said fibrous material comprises a polyamide.

9. The implant anchor apparatus of claim 5, further comprising:
(a) a second tubular section of fibrous material;
(b) said second tubular section having an inner surface configured to fit adjacent to said implant;
(c) said fibrous material capable of becoming fibrotically encapsulated after implantation in tissue.

10. An anchor apparatus, comprising:
(a) means for coupling to an implant; and
(b) means for anchoring said implant to tissue by fibrotic encapsulation.

11. The implant anchor apparatus of claim 10, wherein said anchoring means comprises a fibrous portion.

12. The implant anchor apparatus of claim 11, wherein said fibrous portion comprises a fibrous outer surface.

13. The implant anchor apparatus of claim 10, wherein said coupling means comprises an inner surface configured to fit over an implant.

14. The coupling means of claim 13, wherein said coupling means further comprises an adhesive.

15. A method for anchoring an implant, comprising
(a) providing an anchor having a fibrous portion capable of becoming fibrotically encapsulated in tissue;
(b) coupling said anchor to said implant; and
(c) implanting said anchor and said implant in an incision adjacent to said tissue.

16. The method of claim 15, wherein said anchor comprises a tubular component having an inner surface and an outer surface, said fibrous portion associated with said outer surface.

17. The method of claim 16, wherein said coupling said anchor to said implant comprises engaging said implant in a bore of said tubular component.

18. The method of claim 17, wherein said coupling said anchor to said catheter further comprises adhering said inner surface of said tubular component to an outer surface of said implant.

19. The method of claim 15, further comprising fibrotically encapsulating said fibrous portion in said tissue.

20. The method of claim 15, wherein said coupling said anchor to said implant comprises:
(a) coupling a first portion of said anchor to a first section of said implant; and
(b) coupling a second portion of said anchor to a second portion of said implant.
